(19) United States
(12) Patent Application Publication
(10) Pub. No.: US 2008/0045759 A1
(43) Pub. Date: Feb. 21, 2008

(54) METHOD AND APPARATUS FOR STEAM DEALKYLATION OF HYDROCARBONS IN AN OLEFIN PLANT

(75) Inventors: Helmut FRITZ, Muenchen (DE); Volker Goeke, Wolfratshausen (DE)

Correspondence Address: CROWELL & MORING LLP;INTELLECTUAL PROPERTY GROUP, P.O. BOX 14300, WASHINGTON, DC, 20044-4300, US

(73) Assignee: Linde Aktiengesellschaft, Wiesbaden (DE)

(21) Appl. No.: 11/840,883

(22) Filed: Aug. 17, 2007

(30) Foreign Application Priority Data
Aug. 18, 2006 (DE) 10 2006 038 894.1

(51) Int. Cl. C07C 13/20

(52) U.S. Cl. 585/15; 585/402; 585/410; 585/487

(57) ABSTRACT

A method and apparatus for treating a fraction consisting predominantly of hydrocarbons having at least six carbon atoms (C₆₊ fraction) as produced in a plant for generating hydrocarbons from the steam reforming of hydrocarbon-containing feedstock, is disclosed. The C₆₊ fraction is conducted to steam dealkylation following hydration where the usable products benzene and hydrogen are produced.

This application claims the priority of German Patent Document No. 10 2006 038 894.1, filed Aug. 18, 2006, the disclosure of which is expressly incorporated by reference herein.

### BACKGROUND AND SUMMARY OF THE INVENTION

The invention relates to a method for treating a fraction consisting predominantly of hydrocarbons with at least six carbon atoms (C₆₊ fraction) as produced in a plant for generating hydrocarbons from the steam reforming of hydrocarbon-containing feedstock (olefin plant) and an apparatus for carrying out the method.

In an olefin plant for the steam reforming of hydrocarbon-containing feedstock material, the hydrocarbon-containing feedstock is mixed with steam and heated to very high temperatures (approx. 850° C.) for a very short time, whereby the longer-chain hydrocarbons in the feedstock are reformed into shorter-chain hydrocarbons. These shorter-chain hydrocarbons (primarily ethanes) are the principal product of such a plant. In addition, a series of by-products is produced whose relative percentage and composition depend on the composition of the hydrocarbon-containing feedstock.

One of the primary by-products is pyrolysis gasoline. It is highly aromatic (30% benzene, 15% toluene, 20% C8 aromatics), contains many olefins and conjugated diolefins and is separated in the plant from the remaining product stream as a fraction which consists predominantly of hydrocarbons with at least five carbon atoms (C₅₊ fraction). The C₅₊ fraction contains aromatics as economically utilizable components which find a use as starting materials for the synthesis of numerous plastics and to increase the knock resistance of gasoline. According to the prior art, the C₅₊ fraction first undergoes selective hydration, where the diolefins and styrenes are converted into their respective olefins, or ethyl benzenes. Subsequently distillative separation of the C₅₊ fraction takes place into a fraction which contains predominantly hydrocarbons having five carbon atoms and a fraction which contains predominantly hydrocarbons having at least six carbon atoms (C₆₊ fraction). The resulting C₆₊ fraction undergoes hydration to convert and remove components containing sulfur, nitrogen and/or oxygen. The now hydrated C₆₊ fraction is separated, in accordance with the prior art, by distillation into a fraction which contains predominantly hydrocarbons having six carbon atoms and a fraction which contains predominantly hydrocarbons with at least seven carbon atoms (C₇₊ fraction). From the fraction which contains predominantly hydrocarbons with six carbon atoms economically utilizable benzene can be extracted by means of extractive rectification. To increase the benzene yield in accordance with the prior art the C₇₊ fraction undergoes hydro-dealkylation.

A method of this type is described, for example, in WO2005071045. The C₇₊ fraction is brought into contact with hydrogen in the presence of a catalyst at a temperature of 400° C. to 650° C. and a pressure of between 20 and 40 bar, where the hydrogen is present in a molar excess of three to six times the hydrocarbons. Under these conditions, the alkyl groups are split off from the respective alkylated aromatics (such as toluene and xylene) so that benzene and the respective alkenes (for example methane and ethane) form.

The consumption of hydrogen in the hydro-dealkylation of the C₇₊ fraction and the costly extractive rectification of the fraction which contains predominantly hydrocarbons with six carbon atoms has a negative effect on the profitability of this method from the prior art for extracting benzene.

### BRIEF DESCRIPTION OF THE DRAWING

FIG. 1 illustrates an embodiment of an apparatus in accordance with the principles of the present invention.

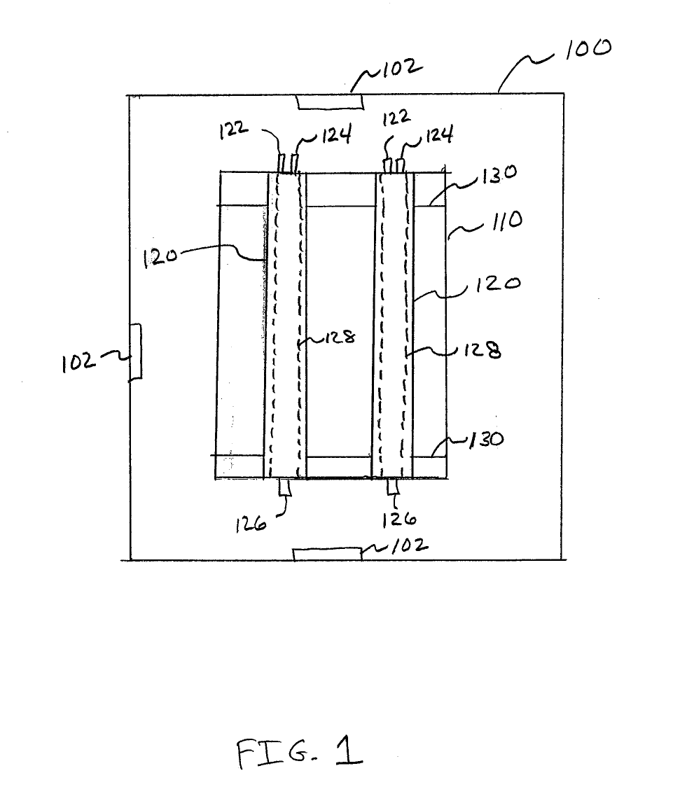

### DETAILED DESCRIPTION OF THE EXEMPLARY EMBODIMENTS

In accordance with the invention, with respect to the method, the C₆₊ fraction is subjected to steam dealkylation, where primarily the two usable product materials benzene and hydrogen are produced along with reaction products such as carbon monoxide and carbon dioxide.

The basic idea of the invention is to perform the dealkylation of the alkylated aromatics by generating benzene with the aid of steam dealkylation. Steam dealkylation requires only inexpensive steam and produces the valuable by-product hydrogen in addition to the desired quality product benzene.

The C₆₊ fraction used in the steam dealkylation contains principally:

a) aromatic hydrocarbons having six to ten carbon atoms,

b) cyclic paraffins (cycloalkenes) having five to ten carbon atoms,

c) iso- and n-paraffins having five to ten carbon atoms,

d) alkenes having six to ten carbon atoms, or

any mixtures of the aforementioned, where the exact composition of the mixture is dependent on the specific hydrocarbon-containing starting material of the olefin plant. A starting material consisting more of shorter-chain hydrocarbons in the steam reforming of the olefin plant has a clearly lower percentage of aromatics in the gas than a starting material containing more longer-chain hydrocarbons. The method in accordance with the invention is suitable for each of the compounds of the C₆₊ fraction described.

The hydrocarbons from the C₆₊ fraction react advantageously with steam in the gas phase with the addition of heat on a solid-bed catalyst. The gaseous C₆₊ fraction is dealkylated by the presence of gaseous water (steam) on a catalyst with the constant addition of heat, whereby the desired products benzene and hydrogen are produced in addition to carbon monoxide, carbon dioxide and additional by-products.

The heat required for dealkylation is preferably generated by the combustion of a starting material with air. It proves to be particularly advantageous to use as well gaseous reaction by-products from the steam dealkylation, in particular carbon monoxide and methane, as the starting material for the combustion with air. A part of the gaseous reaction by-products from the steam dealkylation, in particular carbon monoxide and methane, is combustible and can serve as the starting material for combustion to generate the necessary reaction heat. This saves heating gas and this otherwise unused part of the reaction products is employed usefully.

Following compression in pressure swing adsorption, the gaseous reaction products are expediently separated into gaseous hydrogen and gaseous reaction by-products, in particular carbon monoxide, carbon dioxide and methane. The valuable by-product hydrogen is also present in gaseous form and can be employed much more usefully than in combustion. Through an adsorptive alternating pressure process following compression, the hydrogen can easily be separated from the combustible gaseous reaction by-products which can serve as starting material in combustion.

The flue gases generated during combustion are advantageously cooled by means of a heat exchanger while heating the starting materials for steam dealkylation. Through the use of the heat from the flue gases to preheat the starting materials (C₆₊ fraction and steam) for steam dealkylation, the heat to be brought in which is necessary to maintain the required temperatures for the dealkylation reaction is reduced. In this way an economical use of energy resources is achieved.

The C₆₊ fraction and the steam are advantageously directed past the solid catalyst in pipes, preferably from top to bottom, where the catalyst is situated on the inside of the pipes. Heat is expediently supplied to the pipes from outside. The heat required for the dealkylation reaction is preferably transferred to the pipe by electromagnetic radiation, thermal radiation and/or convection. The actual dealkylation reaction takes place in the interior of the pipes where the catalyst is situated. The two constituents in the reaction (C₆₊ fraction and steam) are directed from top to bottom through the pipes filled with the catalyst. The heat required for the dealkylation reaction is generated outside the pipes and transferred by way of the mechanisms named to the pipe from where the heat is transferred into the interior of the pipe, to the site of the reaction, by means of heat conduction or convection.

Preferably a solid-bed catalyst of a porous carrier material is used, in particular γ-Al₂O₃, MgAl spinel and/or Cr₂O₃, and an active component on the surface of the carrier material, in particular Rh with 0.1-1.0% loading by weight and/or Pd with 0.2-2.0% loading by weight.

The steam dealkylation is advantageously performed at a temperature of 400° C. to 600° C., preferably 450° C. to 550° C., particularly preferably 480° C. to 520° C. and at a pressure of 1 to 15 bar, preferably 1.2 to 10 bar, particularly preferably 1.5 to 8 bar.

The steam dealkylation is expediently performed at a molar quotient of steam to hydrocarbons which lies in the range from 1 to 20, preferably from 2 to 15, when it enters the reactor. In another embodiment of the invention, the steam dealkylation is performed at a molar quotient of steam to hydrocarbons which lies in the range from 3 to 12, preferably from 5 to 10, when it enters the reactor. Generally the steam dealkylation is performed with a molar excess of water, where the exact ratio in the different embodiments of the invention depends on the precise composition of the C₆₊ fraction.

It proves advantageous to subject the C₆₊ fraction to a process to convert dienes and styrenes before steam dealkylation, where specifically hydrating methods which consume hydrogen are employed. It is similarly advantageous to subject the C₆₊ fraction to a process to convert and remove components containing sulfur, nitrogen and/or oxygen before steam dealkylation, where specifically methods which consume hydrogen are also employed. By employing the hydrating methods, the diolefins present in the C₆₊ fraction are converted into their corresponding olefins, just as components containing sulfur, nitrogen and oxygen can be converted and removed. Deactivation of the catalyst is reduced and the life of the catalyst is clearly increased.

The reaction products from steam dealkylation are preferably cooled and separated in a 3-phase separation into gaseous reaction products, hydrocarbons and water. The reaction products coming from steam dealkyation contain not only the desired quality products benzene and hydrogen but also reaction products such as carbon monoxide and carbon dioxide and reaction by-products. To obtain the desired quality products, the reaction products must be separated. This is done by way of a 3-phase separation of the cooled reaction products into gaseous reaction products, in particular hydrogen, carbon monoxide, carbon dioxide and methane, into hydrocarbons, in particular benzene, and into water.

The hydrogen generated in the steam dealkylation of the C₆₊ fraction is expediently fed completely or partially into the starting material for the hydrogen-consuming processes. The hydrogen generated in steam dealkylation can be used entirely or partially for the hydrogen-consuming processes described in the previous section so that the need for hydrogen to be supplied externally is minimized.

In another embodiment of the invention, the hydrogen generated in the steam dealkylation of the C₆₊ fraction is taken as the starting material for any number of other hydrogen-consuming hydration processes for products and by-products from the olefin plant, in particular to saturate fractions consisting predominantly of hydrocarbons having four or more carbon atoms. The hydration of the C₆₊ fraction is not the only hydrogen-consuming process in an olefin plant. Hydration processes are necessary for the primary products of the olefin plant for which the hydrogen generated in steam dealkylation can likewise be used.

In a further embodiment of the invention, the hydrogen generated in the steam dealkylation of the C₆₊ fraction is taken to an oil refinery as starting material.

The reduction of the sulfur content in the C₆₊ fraction to below 10 ppm, preferably to below 3 ppm, particularly preferably to below 1 ppm, before steam dealkylation proves advantageous for a good yield of the desired reaction product benzene from steam dealkylation.

Preferably the benzene is separated from the hydrocarbons of the reaction products through rectification. Following rectification, the benzene advantageously undergoes adsorptive fine cleaning to dry and remove the trace components, where the benzene is directed across an adsorbent on which the trace components, as opposed to benzene, are adsorbed. By applying the inventive method, the benzene can be extracted from the reaction products by simple rectification and processed further or marketed. Expensive extraction or extractive rectification as when applying a process in accordance with the prior art is not necessary, thus reducing investment and process costs.

Advantageously all components in the C₆₊ fraction boiling close to benzene in distillation or forming azeotropes are converted in steam dealkylation. All reaction products from rectification which are heavier boiling than benzene, consisting predominantly of non-converted starting materials from the steam dealkylation, are expediently returned to steam dealkylation as starting material by way of optional hydration. In another embodiment of the invention, all reaction products from rectification which are heavier boiling than benzene, consisting predominantly of non-converted starting materials from steam dealkylation are returned for hydration of the C₆₊ fraction or for hydration of a fraction consisting predominantly of hydrocarbons having at least five carbon atoms prior to steam dealkylation. By returning the non-converted starting materials for hydration or for steam dealkylation, circulation is achieved without losing valuable starting materials.

Concerning the apparatus, the object is achieved by the apparatus comprising an oven 100 with a furnace 110 and pipes 120 located in the furnace. The actual steam dealkylation takes place in the pipes which in turn are located in the furnace of the oven where the heat required for steam dealkylation can be generated.

The pipes are advantageously installed vertically in the furnace and have heat expansion compensating elements 130 at the lower and/or upper end. The heat expansion compensating elements at the lower and/or upper end of the vertical pipes prevent mechanical stress from temperature differences which can lead to increased wear of the pipes.

Each pipe expediently has a supply for the C₆₊ fraction and the steam, 122, 124, respectively, and an outlet 126 for the reaction products.

It similarly proves advantageous that each pipe is filled on the inside with a catalyst 128, where the catalyst consists of a porous carrier material, in particular γ-Al₂O₃, MgAl spinel and/or Cr₂O₃ and an active component on the surface of the carrier material, in particular Rh with 0.1-1.0% loading by weight and/or Pd with 0.2.-2.0% loading by weight.

Preferably the oven has at least one burner 102 on the wall, the ceiling and/or the floor. The pipes are expediently suitable for an internal pressure of 1 to 15 bar, preferably 1.2 to 10 bar, particularly preferably 1.5 to 8 bar, and for use in an oven with flame temperatures of up to 1400° C.

The present invention is successful specifically in creating an economical alternative to the prior art for treating a C₆₊ fraction. Through the application of the inventive method and the inventive apparatus, the valuable by-product hydrogen is generated in addition to the usable product benzene.

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the appended claims and equivalents thereof.

What is claimed is:

1. A method for treating a fraction consisting predominantly of hydrocarbons having at least six carbon atoms (C₆₊ fraction) as produced in a plant for generating hydrocarbons from steam reforming of hydrocarbon-containing feedstock, wherein the C₆₊ fraction undergoes steam dealkylation, wherein two usable product materials benzene and hydrogen are produced in addition to reaction products such as carbon monoxide and carbon dioxide.

2. The method according to claim 1, wherein the C₆₊ fraction contains:
a) aromatic hydrocarbons having six to ten carbon atoms;
b) cyclic paraffins (cycloalkanes) having five to ten carbon atoms;
c) iso- and n-paraffins having five to ten carbon atoms;
d) alkenes having six to ten carbon atoms; or
any mixture of the aforementioned.

3. The method according to claim 1, wherein the hydrocarbons from the C₆₊ fraction react with water in a gas phase with addition of heat to a solid catalyst.

4. The method according to claim 1, wherein heat required for the dealkylation reaction is generated by combustion of a starting material with air.

5. The method according to claim 1, wherein gaseous reaction products from the steam dealkylation following compression are separated by way of pressure swing adsorption into gaseous hydrogen and gaseous reaction by-products, in particular carbon monoxide, carbon dioxide and methane.

6. The method according to claim 5, wherein the gaseous reaction by-products from the steam dealkylation, in particular carbon monoxide and methane, are used as a starting material for combustion with air.

7. The method according to claim 1, wherein flue gases created during combustion are cooled by a heat exchanger while heating starting materials for the steam dealkylation.

8. The method according to claim 1, wherein the C₆₊ fraction and the steam are directed past a solid-bed catalyst in pipes where the catalyst is on an inside of the pipes.

9. The method according to claim 8, wherein heat is brought to the pipes from outside.

10. The method according to claim 9, wherein the heat required for steam dealkylation is transferred by electromagnetic radiation, thermal radiation and/or convection.

11. The method according to claim 1, wherein a solid-bed catalyst of a porous carrier material is used, in particular γ-Al₂O₃, MgAl spinel and/or Cr₂O₃ and an active component on a surface of the carrier material in particular Rh with 0.1-1.0% loading by weight, and/or Pd with 0.2.-2.0% loading by weight.

12. The method according to claim 1, wherein the steam dealkylation is carried out at a temperature of 400° C. to 600° C., preferably 450° C. to 550° C., particularly preferably 480° C. to 520° C.

13. The method according to claim 1, wherein the steam dealkylation is carried out at a pressure of 1 to 15 bar, preferably 1.2 to 10 bar, particularly preferably 1.5 to 8 bar.

14. The method according to claim 1, wherein the steam dealkylation is carried out at a molar quotient of steam to hydrocarbons which is in a range from 1 to 20, preferably from 2 to 15, when it enters a reactor.

15. The method according to claim 1, wherein the steam dealkylation is carried out at a molar quotient of steam to hydrocarbons which is in a range from 3 to 12, preferably from 5 to 10, when it enters a reactor.

16. The method according to claim 1, wherein the C₆₊ fraction prior to the steam dealkylation undergoes a process to convert dienes and styrenes, where in particular hydrating processes which consume hydrogen are used therefor.

17. The method according to claim 1, wherein the C₆₊ fraction undergoes a process prior to the steam dealkylation to convert and remove components containing sulfur, nitrogen and/or oxygen, where in particular hydrating processes which consume hydrogen are used therefor.

18. The method according to claim 1, wherein the reaction products from the steam dealkylation are cooled and separated in a 3-phase separation into gaseous reaction products, hydrocarbons and water.

19. The method according to claim 16, wherein the hydrogen produced in the steam dealkylation of the C₆₊ fraction is fed partially or completely into a starting material for the processes which consume hydrogen.

20. The method according to claim 17, wherein the hydrogen produced in the steam dealkylation of the C₆₊ fraction is fed partially or completely into a starting material for the processes which consume hydrogen.

21. The method according to claim 1, wherein the hydrogen produced in the steam dealkylation of the C₆₊ fraction is fed as starting material to a hydration process of products and by-products from the plant that consumes hydrogen, in particular to a process to saturate fractions consisting predominantly of hydrocarbons having four or more carbon atoms.

22. The method according to claim 1, wherein the hydrogen produced during the steam dealkylation of the C₆₊ fraction is taken to a petroleum refinery as starting material.

23. The method according to claim 1, wherein a sulfur content in the C₆₊ fraction is reduced to below 10 ppm, preferably below 3 ppm, particularly preferably below 1 ppm prior to the steam dealkylation.

24. The method according to claim 1, wherein the benzene is separated from the hydrocarbons by way of rectification of the reaction products.

25. The method according to claim 24, wherein the benzene undergoes absorptive fine cleaning following rectification to dry and remove trace components, where the benzene is directed across an absorbent on which the trace components are adsorbed.

26. The method according to claim 1, wherein components in the C₆₊ fraction boiling close to benzene or forming azeotropes are converted by steam dealkylation.

27. The method according to claim 24, wherein all reaction products from the rectification which are heavier boiling than benzene consisting predominantly of non-converted starting materials from the steam dealkylation are returned by way of an optional hydration to the steam dealkylation as starting material.

28. The method according to claim 24, wherein all reaction products from the rectification which are heavier boiling than benzene consisting predominantly of non-converted starting materials from the steam dealkylation are returned to hydration of the C₆₊ fraction or to hydration of a fraction consisting predominantly of hydrocarbons having at least five carbon atoms prior to steam dealkylation.

29. An apparatus for treating a fraction consisting predominantly of hydrocarbons having at least six carbon atoms (C₆₊ fraction) as produced in a plant for generating hydrocarbons from steam reforming of hydrocarbon-containing feedstock, wherein the apparatus includes an oven with a furnace and pipes located in the furnace.

30. The apparatus according to claim 29, wherein the pipes are mounted vertically in the furnace and have heat expansion compensation elements at a bottom and/or a top end.

31. The apparatus according to claim 29, wherein each pipe has a feed for the C₆₊ fraction and the steam and an outlet for reaction products.

32. The apparatus according to claim 29, wherein each pipe is filled on an inside with a catalyst, where the catalyst consists of a porous carrier material, in particular γ-Al₂O₃, MgAl spinel and/or Cr₂O₃ and an active component on a surface of the carrier material in particular Rh with 0.1-1.0% loading by weight, and/or Pd with 0.2.-2.0% loading by weight.

33. The apparatus according to claim 29, wherein the oven has at least one burner on a wall, a ceiling and/or a floor.

34. The apparatus according to claim 29, wherein the pipes are suitable for an internal pressure of from 1 to 5 bar, preferably 1.2 to 10 bar, particularly preferably 1.5 to 8 bar, and for use in an oven with flame temperatures of up to 1400° C.

35. A method of extracting benzene from a hydrocarbon having at least six carbon atoms, comprising the steps of:
subjecting the hydrocarbon having at least six carbon atoms to steam dealkylation; and
producing benzene from the steam dealkylation.

36. The method according to claim 35, further comprising the step of producing hydrogen from the steam dealkylation.
